(19) United States
(12) Patent Application Publication
(10) Pub. No.: US 2014/0277103 A1
(43) Pub. Date: Sep. 18, 2014

(54) QUICK-CONNECT BUCKLE FOR A TOURNIQUET AND METHODS ASSOCIATED THEREWITH

(71) Applicant: PHIL DURANGO LLC, Fraser, CO (US)

(72) Inventor: Mark ESPOSITO, Fraser, CO (US)

(73) Assignee: PHIL DURANGO LLC, Fraser, CO (US)

(21) Appl. No.: 13/843,792

(22) Filed: Mar. 15, 2013

(51) Int. Cl. A61B 17/132; A44B 11/25

(52) U.S. Cl. CPC A61B 17/1325; A44B 11/25
USPC 606/203; 24/303

(57) ABSTRACT

A quick-connect buckle is provided for a tourniquet, the quick-connect buckle including a dovetail tongue and groove coupling system with an initial coupling position and a final coupling position corresponding to a tensioned configuration. The quick-connect coupler also has application beyond use in tourniquets. Accordingly, the quick-connect coupler can be combined with a strap to form an all-purpose binding device used for a variety of purposes. Methods associated with the quick-connect buckle are also described.

### FIELD

The present invention is related to a medical device, and more particularly to a quick-connect buckle coupling device for a tourniquet.

### BACKGROUND

Loss of blood is a major cause of death in emergency situations in which the injured person is alone or medical assistance is not immediately available. The use of a tourniquet to stop blood loss from an injured arm or leg is a well-known technique for preventing death in these situations.

A person who is alone and suffering a life-threatening injury from a severely wounded limb may have to self-apply a tourniquet to the limb or otherwise die from a loss of blood. If the injured person can apply a tourniquet in a relatively short period of time, he or she can increase his or her likelihood of surviving. Accordingly, there is a need for a tourniquet with structure that assists the injured person, or an aid giver tending to the injured person, with the ability to quickly apply a tourniquet to the injured limb.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of other embodiments.

In accordance with at least one embodiment, a quick-connect buckle is provided for a tourniquet to permit a tourniquet to be easily and quickly applied to an extremity of an injured person, including a trapped extremity (e.g., a person's arm pinned under an object). Accordingly, a quick-connect tourniquet clasp is provided for forming part of a tourniquet and configured for attaching a tourniquet to an extremity of a person, the tourniquet including one or more straps, the quick-connect tourniquet clasp comprising:
a buckle member and a coupling member, the buckle member and the coupling member each adapted for operative association with the one or more straps, the buckle member and coupling member having a complimentary dovetail tongue and groove mating structure, wherein a first magnetic element of the buckle member is adapted for engaging a second magnetic element of the coupling member.

In accordance with at least one embodiment, the complimentary dovetail tongue and groove mating structure include a pair of serrated mating surfaces. In accordance with at least one embodiment, the pair of serrated mating surfaces each form a surface shared by a tongue and groove portion of each of the buckle member and coupling member. In accordance with at least one embodiment, the pair of serrated mating surfaces are not perpendicular relative to a bottom of the buckle member or the coupling member. In accordance with at least one embodiment, the complimentary dovetail tongue and groove mating structure comprise an over and under configuration, the buckle tongue and the buckle groove overlying the coupling groove and coupling tongue, respectively.

An integrated buckle and tourniquet coupler forming part of a tourniquet and adapted for cooperating with one or more straps of the tourniquet is also provided, the integrated buckle and tourniquet coupler comprising:

(a) a buckle member including:
one or more openings and a weaving bar adapted for receiving the one or more straps; and
a buckle tongue and a buckle groove, the buckle tongue including a first magnetic element; and

(b) a coupling member including:
an opening and a bar adapted for receiving the one or more straps; and
a coupling tongue and a coupling groove, the coupling groove including a second magnetic element adapted for engaging the first magnetic element, wherein the coupling groove is sized to receive the buckle tongue, and wherein the coupling tongue is sized to be received by the buckle groove.

In accordance with at least one embodiment, a first interior tongue surface of the buckle tongue is sloped to engage a similarly sloped second interior tongue surface of the coupling tongue. In accordance with at least one embodiment, the first interior tongue surface of the buckle tongue is serrated and is adapted to frictionally engage serrations on the second interior tongue surface of the coupling tongue. In accordance with at least one embodiment, the buckle tongue resides in at least two positions when placed in the coupling groove, the at least two positions comprising an initial coupling position and a final coupling position, wherein when in the final coupling position, the buckle tongue resides in contact with the coupling tongue.

In addition to the foregoing, an embodiment of a tourniquet featuring an integrated buckle and tourniquet coupler is also provided, the tourniquet comprising;

one or more straps;

a windlass operatively associated with at least one strap of the one or more straps;

a bendable base interconnected to the one or more straps;

a hooked catch for securing the windlass from rotating; and

a quick-connect tourniquet clasp including a buckle member and a coupling member, the buckle member and the coupling member each attached to a portion of the one or more straps, the buckle member and coupling member having a complimentary dovetail tongue and groove mating structure, wherein a first magnetic element of the buckle member is adapted for engaging a second magnetic element of the coupling member.

In accordance with at least one embodiment, the complimentary dovetail tongue and groove mating structure include a pair of serrated mating surfaces. In accordance with at least one embodiment, a first surface of the pair of serrated mating surfaces is associated with the buckle member, and wherein a second surface of the pair of serrated mating surfaces is associated with the coupling member. In accordance with at least one embodiment, the pair of serrated mating surfaces are not perpendicular relative to a bottom of the buckle member or the coupling member. In accordance with at least one embodiment, the complimentary dovetail tongue and groove mating structure comprise an over and under configuration, the buckle tongue and the buckle groove overlying the coupling groove and coupling tongue, respectively.

The quick-connect coupler also has application beyond use in tourniquets. The quick-connect coupler can be combined with a strap to form an all-purpose binding device used for a variety of purposes. Accordingly, a quick-connect coupler for use with a strap is provided, comprising:
a buckle member and a coupling member, the buckle member and the coupling member each adapted for operative association with the strap, the buckle member and coupling member having a complimentary dovetail tongue and groove mating structure, wherein a first magnetic element of the buckle member is adapted for engaging a second magnetic element of the coupling member.

In at least one embodiment, the complimentary dovetail tongue and groove mating structure include a pair of serrated mating surfaces. In at least one embodiment, the a first surface of the pair of serrated mating surfaces is associated with the buckle member, and wherein a second surface of the pair of serrated mating surfaces is associated with the coupling member. In at least one embodiment, the pair of serrated mating surfaces are not perpendicular relative to a bottom of the buckle member or the coupling member. In at least one embodiment, the complimentary dovetail tongue and groove mating structure comprise an over and under configuration, the buckle tongue and the buckle groove overlying the coupling groove and coupling tongue, respectively. In at least one embodiment, at least one of the first magnetic element and second magnetic element are enclosed in plastic.

As used herein, the “initial coupling position” means the position of the coupling member and the buckle member when initially joined. In addition, as used herein, the “final coupling position” means the position of the coupling member and buckle member after the tensioning mechanism (e.g., windlass) of the tourniquet has been used to at least partially tighten the tourniquet.

Various components are referred to herein as “operably associated.” As used herein, “operably associated” refers to components that are linked together in operable fashion, and encompasses embodiments in which components are linked directly, as well as embodiments in which additional components are placed between the two linked components.

The word “exemplary” is used herein to mean serving as an example, instance, or illustration. Any aspect described herein as “exemplary” is not necessarily to be construed as preferred or advantageous over other aspects or designs.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages and features of the one or more present inventions, a more particular description of the one or more present inventions is rendered by reference to specific embodiments thereof which are illustrated in the appended drawings. It should be appreciated that these drawings depict only typical embodiments of the one or more present inventions and are therefore not to be considered limiting of its scope. The one or more present inventions are described and explained with additional specificity and detail through the use of the accompanying drawings in which:

FIG. 1A is a perspective of a tourniquet incorporating an embodiment of an integrated buckle and tourniquet coupler;

FIG. 1B is another perspective view of the tourniquet shown in FIG. 1;

FIG. 2A is a bottom perspective view of the buckle member and coupling member of an integrated buckle and tourniquet coupler in a separated orientation with tourniquet straps shown;

FIG. 2B is a bottom perspective view of the buckle member and coupling member shown in FIG. 2A, again with tourniquet straps shown;

FIG. 3A is a top plan view of the buckle member;

FIG. 3B is a bottom perspective view of the buckle member without a magnetic element insert;

FIG. 3C is a bottom plan view of the buckle member with a magnetic element insert;

FIG. 3D is a bottom perspective view of the buckle member with a magnetic element insert;

FIG. 4A is a top perspective view of the coupling member without a magnetic element insert;

FIG. 4B is another top perspective view of the coupling member without a magnetic element insert;

FIG. 4C is a top perspective view of the coupling member with a magnetic element insert;

FIG. 4D is another top perspective view of the coupling member with a magnetic element insert;

FIG. 5A is side elevation view of the buckle member aligned with the coupling member;

FIG. 5B is perspective view of the buckle member aligned with the coupling member;

FIG. 6A is side elevation view of the buckle member in an initial coupling position with the coupling member;

FIG. 6B is perspective view of the buckle member in an initial coupling position with the coupling member;

FIG. 7A is side elevation view of the buckle member in a final coupling position with the coupling member;

FIG. 7B is perspective view of the buckle member in a final coupling position with the coupling member;

FIG. 8 is a top plan view of another embodiment of a coupling member;

FIG. 9A is side elevation view of the buckle member aligned with the embodiment of the coupling member shown in FIG. 8;

FIG. 9B is a side elevation view of the buckle member in an initial coupling position with the embodiment of the coupling member shown in FIG. 8;

FIG. 9C is side elevation view of the buckle member in a final coupling position with the embodiment of the coupling member shown in FIG. 8;

FIG. 10 is a perspective view of another embodiment of the one or more present inventions.

The drawings are not necessarily to scale.

### DETAILED DESCRIPTION

One or more embodiments of the one or more inventions described herein are directed to a quick-connect buckle for a tourniquet. In accordance with at least one embodiment, a quick-connect buckle is provided for a tourniquet, the quick-connect buckle including a dovetail tongue and groove coupling system with an initial coupling position and a final coupling position corresponding to a tensioned configuration.

Referring now to FIGS. 1A and 1B, an example of a tourniquet 100 possessing an integrated buckle and tourniquet coupler 200 is shown. In addition to including an integrated buckle and tourniquet coupler 200, which is described in detail below, a tourniquet 100 that is suitable for incorporating an integrated buckle and tourniquet coupler 200 may also include (but is not required to include) an outer sleeve 104, an inner strap 108 that is at least partially located within the outer sleeve 104, a base 112 with openings and weaving bars 114 for receiving at least one of the outer strap 104 and the inner sleeve 108, and a tightening mechanism such as a windlass 116. Although connected to a portion of the outer sleeve 104, such as its distal end, the inner strap 108 is able to slide within the outer sleeve 104 when the inner strap 108 is tightened by rotating the windlass 116 to which the inner strap 108 is attached. Once the inner strap 108 is tightened sufficiently to occlude blood flow within the injured limb, the windlass 116 may be secured by placing a portion of the windlass 116 into contact with a securing mechanism 120, such as a hooked catch 124.

An example tourniquet for incorporating an integrated buckle and tourniquet coupler 200 is described in U.S. Pat. No. 7,892,253, the contents of which are incorporated herein by reference. The foregoing patent further describes the cooperation of an outer sleeve, inner strap, and windlass, as well as a buckle which can be replaced using the integrated buckle and tourniquet coupler 200 described below. As those skilled in the art will appreciate, the tourniquet structure described above is but one example of the type of tourniquet that may incorporate the novel integrated buckle and tourniquet coupler 200 described and claimed herein. Accordingly, it is to be understood that the integrated buckle and tourniquet coupler 200 may be used with a variety of tourniquets having different designs and configurations, including tourniquets that do not include an outer sleeve 104 and an inner strap 108. For example, the integrated buckle and tourniquet coupler 200 can be used in a tourniquet that includes a strap that is pulled through a buckle member 204 of the integrated buckle and tourniquet coupler 200, but where the strap does not reside within an outer sleeve.

Referring now to FIGS. 2A and 2B, an embodiment of an integrated buckle and tourniquet coupler 200 is illustrated in a separated configuration. The integrated buckle and tourniquet coupler 200 includes a buckle member 204 and a coupling member 208. The buckle member 204 and coupling member 208 include complimentary coupling structure comprising dovetail tongue and groove elements that assist in first aligning the buckle member 204 with the coupling member 208, and then maintaining a secured coupling engagement after the tightening mechanism of the tourniquet (e.g., a windlass) is used to tighten the strap of the tourniquet.

Referring still to FIGS. 2A and 2B, the buckle member 204 includes a buckle tongue 212 and buckle groove 216, and the coupling member 208 includes a coupling tongue 220 and coupling groove 224. The buckle tongue 212 of the buckle member 204 is sized to be received by the coupling groove 224 of the of the coupling member 208. Similarly, the coupling tongue 220 of the coupling member 208 is sized to be received by the buckle groove 216 of the of the buckle member 204.

Referring still to FIGS. 2A and 2B, and in accordance with at least one embodiment, the buckle member 204 and the coupling member 208 preferably include a magnetic feature for assisting with the initial coupling of the buckle member 204 to the coupling member 208. More particularly, a first magnetic element 228 is located in the buckle member 204 and a second magnetic element 232 is located in the coupling member 208. As seen in FIG. 2A, the first magnetic element 228 of the buckle member 204 may be positioned in the buckle tongue 212, and as seen in FIG. 2B, the second magnetic element 232 of the coupling member 208 may be positioned in the coupling groove 224. This configuration is particularly advantageous because when applying the tourniquet, the coupling member 208 can be initially positioned and then the buckling member 204 brought into contact with the coupling member 208. During this process, the buckle tongue 212 is advanced over the coupling groove 224 per arrows A₁ of both FIGS. 2A and 2B. After making initial contact, the buckle member 204 is held to the coupling member 208 by the magnetic force of the first magnetic element 228 cooperating with the second magnetic element 232.

The first and second magnetic elements 228 and 232 may both be actual magnets, or one of the first and second magnetic elements 228 and 232 may be an actual magnet with the other a ferromagnetic material suitable for interacting with the magnet. Thus, as used herein a “magnetic element” means either a magnet or material susceptible to interaction with a magnet. An advantage to using two magnets is that the magnetic force may be stronger; however, care must be taken during manufacture of the integrated buckle and tourniquet coupler 200 to ensure that the poles of the two magnets are properly aligned to mate with each other and not repel one another. Accordingly, while the magnetic coupling force may not be as strong for use of a single magnet and a magnetic element comprising a ferromagnetic material as compared to two magnets (assuming uniform strength magnets for both scenarios), the manufacture process is simplified and prevents possible manufacture of a tourniquet having a buckle member 204 that is repelled by its coupling member 208. Thus, in one preferred embodiment, the buckle member 204 includes an actual magnet as the first magnetic element 228, and the coupling member 208 includes a ferromagnetic material as the second magnetic element 232.

Referring now to FIGS. 3A-3D, detail views of buckle member 204 are shown. Buckle member 204 includes openings 304 and a weaving bar 308 for receiving the tourniquet strap to which is it connected. (See FIGS. 1A and 1B for illustration of the outer sleeve 104 woven through buckle member 204.)

FIG. 3B illustrates the underside of buckle member 204. A first receptacle 312 for the first magnetic element 228 is present in the buckle tongue 212 depicted in FIG. 3B. Accordingly, and by way of example and not limitation, the buckle member 204 may be manufactured using plastic and an injection mold process, such as by using a nylon composite material, wherein the first receptacle 312 is formed along with the other structure of the buckle member 204. Alternatively, and as discussed in further detail below, the first magnetic element 228 may be molded into the buckle member 204 when manufacturing the buckle member 204.

Referring to FIGS. 3B and 3D, the buckle tongue 212 includes a distal tongue surface 316, a bottom tongue surface 320 and interior tongue surface 324. As can be seen in FIGS. 3B and 3D (and as best seen in FIG. 5A), the distal tongue surface 316 extends down the distal end of the buckle member 204 and is preferably sloped.

The angles between the surfaces of the tongue and groove features of the integrated buckle and tourniquet coupler 200 assist with engaging the buckle member 204 to the coupling member 208. Accordingly, the interior tongue surface 324 is preferably sloped at an angle θ of between about 60 to 80 degrees relative to the bottom tongue surface 320. More preferably, the angle θ between the interior tongue surface 324 and the bottom tongue surface 320 is between about 72 to 78 degrees, and more preferably yet, the angle θ between the interior tongue surface 324 and the bottom tongue surface 320 is about 75 degrees.

At least a portion of the interior tongue surface 324 preferably includes serrations 328 (for engaging serrations on a surface of the coupling member as described below). As depicted in FIGS. 3B and 3D, the entire interior tongue surface 324 may include serrations 328.

Referring now to FIG. 3D, after manufacture of the plastic portion of the buckle member 204, the first receptacle 312 may be fitted with the first magnetic element 228. The first magnetic element 228 may be secured within the first receptacle 312, such as by gluing the first magnetic element 228 into the first receptacle 312, or by other manufacturing techniques, such as sizing the first magnetic element 228 to form a friction fit within the first receptacle 312 when pressed into position. Alternatively, the first magnetic element 228 may be incorporated into the molding process. By way of example, the first magnetic element 228 may be molded into the plastic of the buckle member 204 with its surface exposed adjacent the bottom tongue surface 320 of the buckle tongue 212. Yet another alternative is to mold the first magnetic element 228 into the plastic of the buckle member 204 with its surface covered by a thin film of the plastic, such that no part of the first magnetic element 228 is exposed. So for example, the bottom tongue surface 320 would not include an exposed surface of the first magnetic element 228. By molding the first magnetic element 228 within the buckle member 204 so that it is covered by a thin film of plastic, the first magnetic element 228 is less likely to attract ferrous material, and thereby stay cleaner for possible future use.

Referring now to FIGS. 4A-4D, detail views of coupling member 208 are shown. Coupling member 208 includes opening 404 and a bar 408 for receiving the tourniquet strap to which is it connected. (As with buckle member 204, see FIGS. 1A and 1B for illustration of the outer sleeve 104 woven through coupling member 208.)

FIGS. 4A and 4B illustrate the top of coupling member 208. A second receptacle 412 for the second magnetic element 232 is present in the coupling groove 224. Accordingly, as with buckle member 204, and by way of example and not limitation, the coupling member 208 may be manufactured using plastic and an injection mold process, such as by using a nylon composite material, wherein the second receptacle 412 is formed along with the other structure of the coupling member 208. Alternatively, and as discussed in further detail below, the second magnetic element 232 may be molded into the coupling member 208 when manufacturing the coupling member 208.

Still referring to FIGS. 4A-4D, the coupling tongue 220 includes a distal tongue surface 416, a top tongue surface 420 and an interior tongue surface 424. As can be seen in FIGS. 4A and 4C (and as best seen in FIG. 5A), the distal tongue surface 416 extends down the distal end of the coupling member 208. As with the distal tongue surface 316 of the buckle member 204, the distal tongue surface 416 of the coupling member 208 is preferably sloped.

As noted above, the angles between the surfaces of the tongue and groove features of the integrated buckle and tourniquet coupler 200 assist with engaging the buckle member 204 to the coupling member 208. Accordingly, the interior tongue surface 424 of the coupling member 208 is preferably sloped at an angle θ of between about 60 to 80 degrees relative to the bottom groove surface 414 of the coupling member 208. More preferably, the angle θ between the interior tongue surface 424 and the bottom groove surface 414 is between about 72 to 78 degrees, and more preferably yet, the angle θ between the interior tongue surface 324 and the bottom groove surface 414 is about 75 degrees.

At least a portion of the interior tongue surface 424 preferably includes serrations 428 for engaging the serrations 328 on the interior tongue surface 324 of the buckle member 204. As depicted in FIGS. 4A-4D, the entire interior tongue surface 424 may include serrations 428.

Referring now to FIGS. 4C and 4D, after manufacture of the plastic portion of the coupling member 208, the second receptacle 412 may be fitted with the second magnetic element 232. As with the first magnetic element 228, the second magnetic element may be secured within the second receptacle 412 of the bottom groove surface 414, such as by gluing the second magnetic element 232 into the second receptacle 412, or by other manufacturing techniques, such as sizing the second magnetic element 232 to form a friction fit within the second receptacle 412 when pressed into position. Alternatively, the second magnetic element 232 may be incorporated into the molding process. By way of example, the second magnetic element 232 may be molded into the plastic of the coupling member 208 with its surface exposed adjacent the bottom groove surface 414 of the coupling groove 224. Yet another alternative is to mold the second magnetic element 232 into the plastic of the coupling member 208 with its surface covered by a thin film of the plastic, such that no part of the second magnetic element 232 is exposed. So for example, the bottom groove surface 414 would not include an exposed surface of the second magnetic element 232. By molding the second magnetic element 232 within the coupling member 208 so that it is covered by a thin film of plastic, the second magnetic element 232 is less likely to attract ferrous material, and thereby stay cleaner for possible future use.

Referring now to FIGS. 5A and 5B, to join the buckle member 204 to the coupling member 208, the buckle tongue 212 of the buckle member 204 is placed in alignment with the coupling groove 224 of the coupling member 208, and the buckle groove 216 of the buckle member 204 is placed in alignment with the coupling tongue 220 of the coupling member 208. As can be seen in FIG. 5A, the interior tongue surface 324 of the buckle tongue 212 is substantially parallel and is situated for adjacent alignment with the interior tongue surface 424 of the coupling member 208. Thus, when aligned, the buckle tongue 212 can slide into the coupling groove 224.

Referring now to FIGS. 6A and 6B, the buckle member 204 is shown in an initial coupling position with the coupling member 208. More particularly, the buckle tongue 212 has been slid into the coupling groove 224. In this initial coupling position, the first magnetic element 228 engages the second magnetic element 232 and holds the buckle member 204 to the coupling member 208 so that the operator of the tourniquet is able to rotate the windlass 116 to apply tension to the strap 108 of the tourniquet. The sloped surface of the buckle tongue 312 and coupling tongue 220 serve to assist in preventing the buckle member 204 from disengaging the coupling member 204.

Referring now to FIGS. 7A and 7B, the buckle member 204 is shown in a final coupling position with the coupling member 208. More particularly, the buckle member 204 has been pulled relative to the coupling member 208 by tension being applied through the strap, such as by pulling the strap when placing the tourniquet on the injured limb (for example, when pulling outer sleeve 104 when applying the tourniquet 100 to the injured limb). In this tensioned configuration, the interior tongue surface 324 of the buckle member 204 has contacted the interior tongue surface 424 of the coupling member 208. Of course, further tension is provided by the strap when tightening the strap as a result of rotating the windlass (for example, when tightening strap 108 using windlass 116). In the tensioned configuration, serrations 328 on the interior tongue surface 324 of the buckle member 204 engage the serrations 428 on the interior tongue surface 424 of the coupling member 208, thereby assisting in securing together the buckle member 204 to the coupling member 208.

Referring now to FIG. 8, and in accordance with at least one embodiment, coupling member 208 may include closed lateral sides 804 along the coupling groove 224, with sloping interior sidewalls 808. Preferably, the angle between the bottom groove surface 414 and the sloping interior sidewalls 808 is between about 100 to 130 degrees, and more preferably, between about 110 to 120 degrees. The sloping interior sidewalls 808 assist in aligning the buckle tongue 212 with the coupling groove 224, thereby further assisting in aligning the first magnetic element 228 of the buckle member 204 with the second magnetic element of the coupling member 208. In addition, the closed lateral sides 804 assist in maintaining buckle member 204 in engagement with the coupling member 208 by preventing lateral migration of the buckle tongue 212 from the coupling groove 224 if the tourniquet is jarred, such as in carrying a wounded soldier fitted with the tourniquet.

Referring now to FIG. 9A, to join the buckle member 204 to the coupling member 208, the buckle tongue 212 of the buckle member 204 is initially placed overlying alignment with the coupling groove 224 of the coupling member 208, and the buckle groove 216 of the buckle member 204 is placed in alignment with the coupling tongue 220 of the coupling member 208.

Referring now to FIG. 9B, the buckle member 204 is shown in an initial coupling position with the coupling member 208. Here, the buckle tongue 212 has been slid into the coupling groove 224. In this initial coupling position, the first magnetic element 228 engages the second magnetic element 232 and holds the buckle member 204 to the coupling member 208 so that the operator of the tourniquet is able to rotate the windlass 116 to apply tension to the strap 108 of the tourniquet. Along with the first and second magnetic elements 228 and 232, the closed lateral sides 804 and the sloping interior sidewalls 808 of the coupling groove assist in maintaining the buckle tongue 212 within the coupling groove 224.

Referring now to FIG. 9C, the buckle member 204 is shown in a final coupling position with the coupling member 208. More particularly, the buckle member 204 has been pulled relative to the coupling member 208 by tension being applied through the strap, such as by pulling the strap when placing the tourniquet on the injured limb (for example, when pulling outer sleeve 104 when applying the tourniquet 100 to the injured limb). Again, the closed lateral sides 804 assist in maintaining buckle member 204 in engagement with the coupling member 208 by preventing lateral migration of the buckle tongue 212 from the coupling groove 224.

In use, an operator of a tourniquet featuring an integrated buckle and tourniquet coupler 200 places the coupling member 208 on the extremity and wraps the tourniquet around the extremity, or otherwise positions a portion of the tourniquet around the subject extremity and thereafter brings the buckle member 204 in general coupling alignment with the coupling member 204 by aligning the dovetail tongue and groove structures of the buckle member 204 and coupling member 208. Thereafter, the buckle tongue 212 is inserted into the coupling groove 224 and concurrently the coupling tongue 220 is placed within the buckle groove 216. As the buckle tongue 212 nears the coupling groove 224, the first and second magnetic elements 228, 232 attract one another or otherwise engage and the buckle member 204 is loosely held within the coupling member 208. Thereafter, the windlass or other tension inducing device connected to the strap of the tourniquet is used to tighten the strap, which in turn, draws the buckle member 204 and coupling member 208 apart, thus forcing the serrated surfaces 328 and 428 of the buckle tongue 212 and coupling tongue 220 to contact each other and interlock. Thereafter, the angled surfaces of the buckle tongue 212 and coupling tongue 220 resist separation of the buckle member 204 from the coupling member 208 because vertical separation is not available. Rather, the buckle member 204 must be drawn circumferentially backwards toward the coupling member 208, which action is not easily achieved since the strap of the tourniquet provides a force opposing unlocking of the buckle member 204 from the coupling member 208.

Referring now to FIG. 10, and as those skilled in the art will appreciate, the combination of the buckle member 204 and coupling member 208 has application to devices other than tourniquets. For example the combination of the buckle member 204 and coupling member 208 form a quick-connection coupler 1004 that can be associated with a strap 1008 and used for bundling objects together or to hold/secure materials to another object, such as a cargo strap type of application. Accordingly, the quick-connect coupler can be combined with a strap to form an all-purpose binding device 1000 used for a variety of purposes.

The one or more present inventions may be embodied in other specific forms without departing from its spirit or essential characteristics. The described embodiments are to be considered in all respects only as illustrative and not restrictive. The scope of the one or more present inventions is, therefore, indicated by the appended claims rather than by the foregoing description. All changes which come within the meaning and range of equivalency of the claims are to be embraced within their scope.

The one or more present inventions, in various embodiments, includes components, methods, processes, systems and apparatus substantially as depicted and described herein, including various embodiments, subcombinations, and subsets thereof. Those of skill in the art will understand how to make and use the one or more present inventions after understanding the present disclosure.

The one or more present inventions, in various embodiments, includes providing devices and processes in the absence of items not depicted and/or described herein or in various embodiments hereof, including in the absence of such items as may have been used in previous devices or processes (e.g., for improving performance, achieving ease and/or reducing cost of implementation).

The foregoing discussion of the one or more present inventions has been presented for purposes of illustration and description. The foregoing is not intended to limit the one or more present inventions to the form or forms disclosed herein. In the foregoing Detailed Description for example, various features of the one or more present inventions are grouped together in one or more embodiments for the purpose of streamlining the disclosure. This method of disclosure is not to be interpreted as reflecting an intention that the claimed one or more present inventions requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the one or more present inventions.

Moreover, though the description of the one or more present inventions has included description of one or more embodiments and certain variations and modifications, other variations and modifications are within the scope of the one or more present inventions (e.g., as may be within the skill and knowledge of those in the art, after understanding the present disclosure). It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without in ding to publicly dedicate any patentable subject matter.

What is claimed is:

1. A quick-connect tourniquet clasp forming part of a tourniquet and configured for attaching the tourniquet to an extremity of a person, the tourniquet including one or more straps, the quick-connect tourniquet clasp comprising:
a buckle member and a coupling member, the buckle member and the coupling member each adapted for operative association with the one or more straps, the buckle member and coupling member having a complimentary dovetail tongue and groove mating structure, wherein a first magnetic element of the buckle member is adapted for engaging a second magnetic element of the coupling member.

2. The quick-connect tourniquet clasp of claim 1, wherein the complimentary dovetail tongue and groove mating structure include a pair of serrated mating surfaces.

3. The quick-connect tourniquet clasp of claim 2, wherein a first surface of the pair of serrated mating surfaces is associated with the buckle member, and wherein a second surface of the pair of serrated mating surfaces is associated with the coupling member.

4. The quick-connect tourniquet clasp of claim 3 wherein the pair of serrated mating surfaces are not perpendicular relative to a bottom of the buckle member or the coupling member.

5. The quick-connect tourniquet clasp of claim 4, wherein the complimentary dovetail tongue and groove mating structure comprise an over and under configuration, the buckle tongue and the buckle groove overlying the coupling groove and coupling tongue, respectively.

6. An integrated buckle and tourniquet coupler forming part of a tourniquet and adapted for cooperating with one or more straps of the tourniquet, the integrated buckle and tourniquet coupler comprising:
(a) a buckle member including:
one or more openings and a weaving bar adapted for receiving the one or more straps; and
a buckle tongue and a buckle groove, the buckle tongue including a first magnetic element; and
(b) a coupling member including:
an opening and a bar adapted for receiving the one or more straps; and
a coupling tongue and a coupling groove, the coupling groove including a second magnetic element adapted for engaging the first magnetic element, wherein the coupling groove is sized to receive the buckle tongue, and wherein the coupling tongue is sized to be received by the buckle groove.

7. The integrated buckle and tourniquet coupler of claim 6, wherein a first interior tongue surface of the buckle tongue is sloped to engage a similarly sloped second interior tongue surface of the coupling tongue.

8. The integrated buckle and tourniquet coupler of claim 7, wherein the first interior tongue surface of the buckle tongue is serrated and is adapted to frictionally engage serrations on the second interior tongue surface of the coupling tongue.

9. The integrated buckle and tourniquet coupler of claim 8, wherein the buckle tongue resides in at least two positions when placed in the coupling groove, the at least two positions comprising an initial coupling position and a final coupling position, wherein when in the final coupling position, the buckle tongue resides in contact with the coupling tongue.

10. A tourniquet comprising;
one or more straps;
a windlass operatively associated with at least one strap of the one or more straps;
a bendable base interconnected to the one or more straps;
a hooked catch for securing the windlass from rotating; and
a quick-connect tourniquet clasp including a buckle member and a coupling member, the buckle member and the coupling member each attached to a portion of the one or more straps, the buckle member and coupling member having a complimentary dovetail tongue and groove mating structure, wherein a first magnetic element of the buckle member is adapted for engaging a second magnetic element of the coupling member.

11. The tourniquet of claim 10, wherein the complimentary dovetail tongue and groove mating structure include a pair of serrated mating surfaces.

12. The tourniquet of claim 11, wherein the pair of serrated mating surfaces each form a surface shared by a tongue and groove portion of each of the buckle member and coupling member.

13. The tourniquet of claim 12, wherein the pair of serrated mating surfaces are not perpendicular relative to a bottom of the buckle member or the coupling member.

14. The tourniquet of claim 12, wherein the complimentary dovetail tongue and groove mating structure comprise an over and under configuration, the buckle tongue and the buckle groove overlying the coupling groove and coupling tongue, respectively.

15. A quick-connect coupler for use with a strap, comprising:
a buckle member and a coupling member, the buckle member and the coupling member each adapted for operative association with the strap, the buckle member and coupling member having a complimentary dovetail tongue and groove mating structure, wherein a first magnetic element of the buckle member is adapted for engaging a second magnetic element of the coupling member.

16. The quick-connect coupler of claim 15, wherein the complimentary dovetail tongue and groove mating structure include a pair of serrated mating surfaces.

17. The quick-connect coupler of claim 16, wherein a first surface of the pair of serrated mating surfaces is associated with the buckle member, and wherein a second surface of the pair of serrated mating surfaces is associated with the coupling member.

18. The quick-connect coupler of claim 17, wherein the pair of serrated mating surfaces are not perpendicular relative to a bottom of the buckle member or the coupling member.

19. The quick-connect coupler of claim 18, wherein the complimentary dovetail tongue and groove mating structure comprise an over and under configuration, the buckle tongue and the buckle groove overlying the coupling groove and coupling tongue, respectively.

20. The quick-connect coupler of claim 15, wherein at least one of the first magnetic element and second magnetic element are enclosed in plastic.
